(19) United States
(12) Patent Application Publication
(10) Pub. No.: US 2019/0195757 A1
(43) Pub. Date: Jun. 27, 2019

(54) DETECTION METHOD FOR HEAVY METAL IONS

(71) Applicant: Industrial Technology Research Institute, Hsinchu (TW)

(72) Inventors: Chun-Chi Lee, Hsinchu City (TW); Shu-Kang Hsu, Hsinchu City (TW); Min-Shan Wu, Yunlin County (TW); Yi-Ting Wang, Hsinchu City (TW); Ting-Ting Chang, Taichung City (TW); Kuan-Foo Chang, Hsinchu City (TW)

(73) Assignee: Industrial Technology Research Institute, Hsinchu (TW)

(21) Appl. No.: 16/226,658

(22) Filed: Dec. 20, 2018

Related U.S. Application Data
(60) Provisional application No. 62/609,288, filed on Dec. 21, 2017.

(30) Foreign Application Priority Data
Aug. 27, 2018 (TW) 107129790

(51) Int. Cl. G01N 1/40; G01N 27/42

(52) U.S. Cl. CPC G01N 1/4005; G01N 33/1813; G01N 2001/4011; G01N 27/42

(57) ABSTRACT

Provided is a detection method for heavy metal ions that includes the following steps. A waste water is flowed through an ion-imprinted polymer tube for adsorbing at least two kinds of target heavy metal ions. The ion-imprinted polymer tube is rinsed to remove a non-target object from the ion-imprinted polymer tube. The target heavy metal ions in the ion-imprinted polymer tube are desorbed by using an acid liquid. An electrochemical method is performed to detect concentrations of the target heavy metal ions.

### CROSS-REFERENCE TO RELATED APPLICATION

This application claims the priority benefit of U.S. provisional application Ser. No. 62/609,288, filed on Dec. 21, 2017, and Taiwan application serial no. 107129790, filed on Aug. 27, 2018. The entirety of each of the above-mentioned patent applications is hereby incorporated by reference herein and made a part of this specification.

### TECHNICAL FIELD

The disclosure relates to a detection method for heavy metal ions in waste water.

### BACKGROUND

In recent years, due to the rapid development of industries such as electroplating, optoelectronics, printed circuit boards, and the semiconductor industry, the issue of heavy metal waste water pollution has become increasingly serious. The heavy metal waste water not only causes serious damage to the human body, but also destroys the environment in which humans live. Therefore, the detection method for heavy metal ions in waste water is very important. However, untreated waste water has an interfering matrix that causes the detected signal to be shifted and suppressed, thus reducing the accuracy of qualitative and quantitative detection results. Therefore, waste water must be processed before testing.

In general, the effect of the matrix is reduced by dilution or standard addition, but if the concentrations of the target heavy metal ions are low, the results will be inaccurate. Another method is to modify the electrode to reduce potential shift. However, the cost for modifying the electrode is high and different modified electrodes are required for different target heavy metal ions, resulting in high cost and more processing steps.

### SUMMARY

The disclosure provides a detection method for heavy metal ions that may solve the issue of signal shift and suppression caused by a matrix of waste water, so as to improve the accuracy of qualitative and quantitative detection results.

A detection method for heavy metal ions of the disclosure includes the following steps. A waste water is flowed through an ion-imprinted polymer tube for adsorbing at least two kinds of target heavy metal ions. The ion-imprinted polymer tube is rinsed to remove a non-target object from the ion-imprinted polymer tube. The target heavy metal ions in the ion-imprinted polymer tube are desorbed by using an acid liquid. An electrochemical method is performed to detect the concentrations of the target heavy metal ions.

Several exemplary embodiments accompanied with figures are described in detail below to further describe the disclosure in details.

### BRIEF DESCRIPTION OF THE DRAWINGS

The accompanying drawings are included to provide further understanding, and are incorporated in and constitute a part of this specification. The drawings illustrate exemplary embodiments and, together with the description, serve to explain the principles of the disclosure.

FIG. 1 is a flowchart of a detection method for heavy metal ions of an embodiment of the disclosure.

FIG. 2 is a schematic of an operation of a detection method for heavy metal ions of an embodiment of the disclosure.

FIG. 3 shows device schematics of a screen-printed tri-electrode plate and a rod-shaped tri-electrode system used in an embodiment of the disclosure.

FIG. 4 is a potential-current diagram of experimental example 1, comparative example 1, and comparative example 2 of the disclosure.

FIG. 5 is a potential-current diagram of experimental example 2, comparative example 3, and comparative example 4 of the disclosure.

### DETAILED DESCRIPTION OF DISCLOSED EMBODIMENTS

In the following detailed description, for purposes of explanation, numerous specific details are set forth in order to provide a thorough understanding of the disclosed embodiments. It will be apparent, however, that one or more embodiments may be practiced without these specific details. In other instances, well-known structures and devices are schematically shown in order to simplify the drawing.

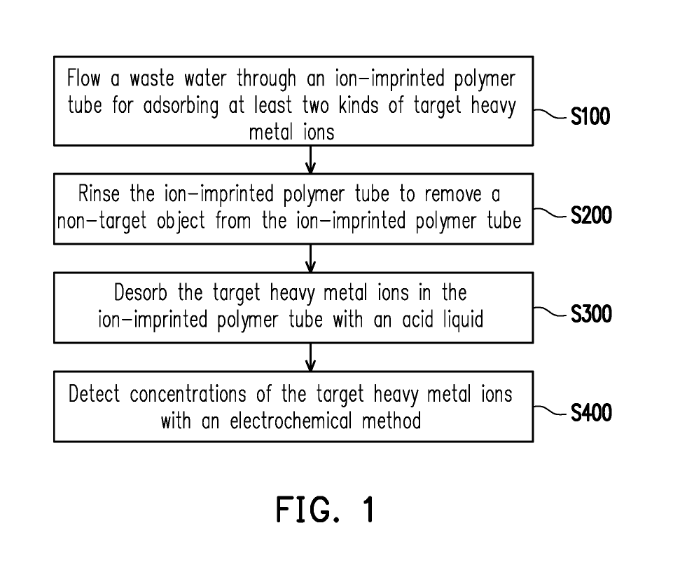

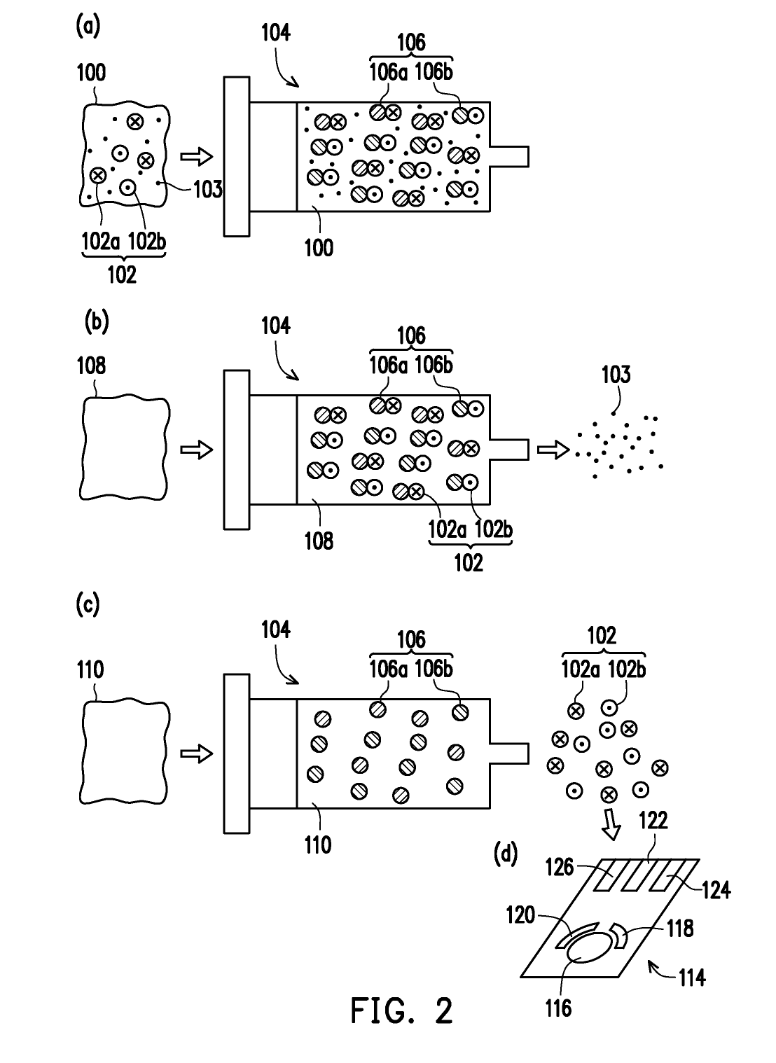

FIG. 1 is a flowchart of a detection method for heavy metal ions of an embodiment of the disclosure. FIG. 2 is a schematic of an operation of a detection method for heavy metal ions of an embodiment of the disclosure.

Referring to FIG. 1 and part (a) of FIG. 2, step S100 is performed to flow a waste water 100 through an ion-imprinted polymer tube 104 for adsorbing at least two kinds of target heavy metal ions 102. The waste water 100 contains the target heavy metal ions 102 and a non-target object 103. In an embodiment, the target heavy metal ions 102 include first target heavy metal ions 102a and second target heavy metal ions 102b, but the disclosure is not limited thereto. In another embodiment, the target heavy metal ions 102 may also include three or more than three kinds of target heavy metal ions.

Moreover, the target heavy metal ions 102 include at least two kinds of lead ions, copper ions, chromium ions, nickel ions, zinc ions, and cadmium ions. The target heavy metal ions 102 may also be other metal ions commonly found in the waste water 100. The non-target object 103 is a matrix of the waste water 100. The non-target object 103 includes non-target heavy metal ions, organic substances, conductive impurities, interferents, or a combination thereof, but the disclosure is not limited thereto.

In an embodiment, the conductivity of the waste water 100 is greater than or equal to about 2,000 microSiemens/cm. More specifically, the conductivity of the waste water 100 is, for example, between about 2,000 microSiemens/cm and 3,000 microSiemens/cm. When stripping voltammetry is used to measure the concentrations of heavy metal ions in the waste water, the peak position and intensity are significantly affected by the conductivity of the waste water, causing signal shift and deformation, which causes error in qualitative and quantitative detection.

In an embodiment, the ion-imprinted polymer tube 104 has at least two kinds of ion-imprinted polymers 106 for adsorbing the at least two kinds of target heavy metal ions 102. In an embodiment, the ion-imprinted polymers 106 include a first ion-imprinted polymer 106a and a second ion-imprinted polymer 106b. The first ion-imprinted polymer 106a and the second ion-imprinted polymer 106b are, for example, uniformly mixed with silica sand and then filled in the ion-imprinted polymer tube 104. In an embodiment, the ion-imprinted polymer tube 104 may also have three or more than three kinds of ion-imprinted polymers for adsorbing three or more than three kinds of target heavy metal ions.

The ion-imprinted polymers 106 are produced by first providing template molecules and then providing functional monomers to bond with the template molecules. Then, a crosslinking agent is added to perform polymerization to produce polymers, and the template molecules are removed from the polymer to obtain ion-imprinted polymers 106 having pores, and the pores have functional groups on the surfaces thereof.

Since the pore structures of the ion-imprinted polymers 106 is the same as those of the originally bonded template molecules, the pores may capture the target heavy metal ions 102 which are similar in structure to the template molecules. Thereafter, the ion-imprinted polymers 106 are bonded to the target heavy metal ions 102 captured by the pores to fix the target heavy metal ions 102. Therefore, the ion-imprinted polymers 106 have a high selectivity for identifying a specific target and adsorbing the specific target, such that different kinds of ion-imprinted polymers 106 may adsorb different kinds of target heavy metal ions 102.

In an embodiment, the pores of the first ion-imprinted polymer 106a and the second ion-imprinted polymer 106b have different structures, and therefore the first ion-imprinted polymer 106a and the second ion-imprinted polymer 106b adsorb different kinds of target heavy metal ions 102. Further, since the structures of the pores of the ion-imprinted polymers 106 are different from that of the non-target object 103, the ion-imprinted polymers 106 do not adsorb the non-target object 103.

It is noted that different kinds of target heavy metal ions 102 in the waste water 100 are simultaneously adsorbed by the ion-imprinted polymer tube 104, and the target heavy metal ions 102 are adsorbed in a single ion-imprinted polymer tube 104. Thus, the disclosure eliminates the need to use more than one ion-imprinted polymer tube 104 to adsorb the target heavy metal ions 102, thereby saving detection steps and costs.

Then, referring to FIG. 1 and part (b) of FIG. 2, step S200 is performed to rinse the ion-imprinted polymer tube 104 to remove the non-target object 103 from the ion-imprinted polymer tube 104. In more detail, since the ion-imprinted polymers 106 are bonded with the target heavy metal ions 102, the target heavy metal ions 102 are adsorbed by the ion-imprinted polymers 106 and are not rinsed out of the ion-imprinted polymer tube 104. The non-target object 103 is not adsorbed by the ion-imprinted polymers 106, and therefore when the ion-imprinted polymer tube 104 is rinsed, the non-target object 103 flows out of the ion-imprinted polymer tube 104.

In an embodiment, a deionized water 108 is used to rinse the ion-imprinted polymer tube 104, although the disclosure is not limited thereto. In an embodiment, the ion-imprinted polymer tube 104 is rinsed using a buffer solution or an organic solution. The buffer solution includes acetic acid/sodium acetate, hydrochloric acid, Britton-Robinson buffer solution, ammonium/ammonium chloride, phosphoric acid, the like or a combination thereof, and the organic solvent may be methanol, ethanol, the like or a combination thereof. Further, any liquid which has good fluidity and does not break the bond between the ion-imprinted polymers 106 and the target heavy metal ions 102 is suitable for rinsing the ion-imprinted polymer tube 104.

Thereafter, referring to FIG. 1 and part (c) of FIG. 2, step S300 is performed to desorb the target heavy metal ions 102 in the ion-imprinted polymer tube 104 with an acid liquid 110. In more detail, the acid liquid 110 weakens the bond between the ion-imprinted polymers 106 and the target heavy metal ions 102, and therefore, the ion-imprinted polymers 106 is unable to continue adsorbing the target heavy metal ions 102. In addition, the smaller the pH of the acid liquid 110, the stronger the acidity, and the better the effect of weakening the bond between the ion-imprinted polymers 106 and the target heavy metal ions 102. The pH of the acid liquid 110 is, for example, less than or equal to about 5, such as between about 0 and about 5. The acid liquid 110 includes sulfuric acid, hydrochloric acid, nitric acid, or a combination thereof, but the disclosure is not limited thereto. The hydrochloric acid has, for example, a pH of about 2. In addition, other compounds which may be used to weaken the bond between the ion-imprinted polymers 106 and the target heavy metal ions 102 are also suitable for use in the disclosure.

In addition, after the target heavy metal ions 102 are desorbed, the ion-imprinted polymer tube 104 may be dried to be used again to adsorb the target heavy metal ions 102, thus achieving a cost-saving effect.

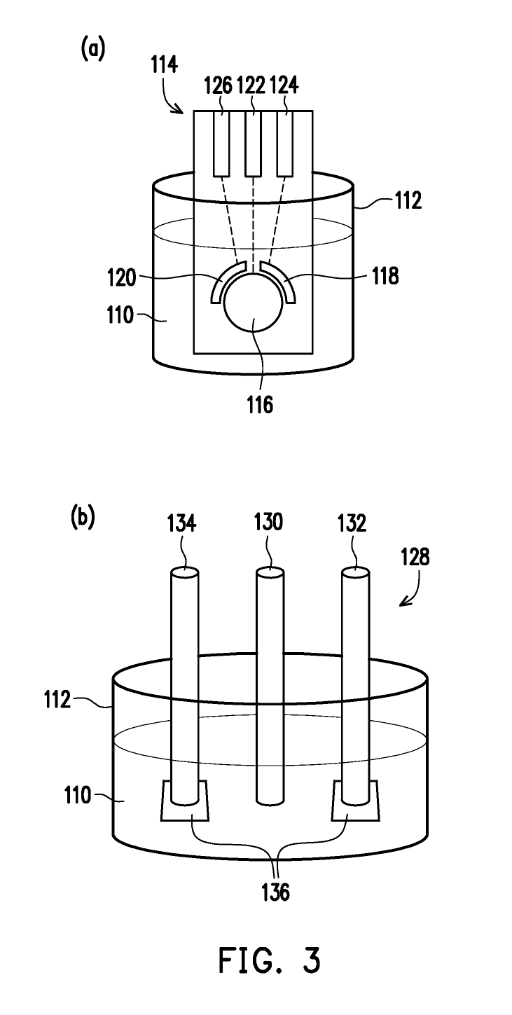

Next, referring to FIG. 1 and FIG. 2(d), step S400 is performed to detect the concentrations of the target heavy metal ions 102 by an electrochemical method. Specifically, after the acid liquid 110 desorbs the target heavy metal ions 102, the acid liquid 110 containing the target heavy metal ions 102 is flowed to a detection container 112 as shown in FIG. 3. Next, an electrochemical method is performed to detect the concentrations of the target heavy metal ions 102. In an embodiment, the electrochemical method used is anodic stripping voltammetry (ASV). In addition, in particular cases (e.g., moderate concentration, and qualitative), cyclic voltammetry alone may be used.

In an embodiment, the anodic stripping voltammetry is performed to detect the concentrations of the target heavy metal ions 102 using a screen-printed tri-electrode plate 114, and a device schematic of the screen-printed tri-electrode plate 114 is shown in part (a) of FIG. 3. The screen-printed tri-electrode plate 114 has a small size, so as to be convenient to carry.

Next, referring to part (a) of FIG. 3, the screen-printed tri-electrode plate 114 has a working electrode 116, a counter electrode 118, and a reference electrode 120. The material of the working electrode 116 includes gold or ruthenium. The material of the counter electrode 118 includes gold, platinum, or carbon. The material of the reference electrode 120 includes silver chloride or silver. In addition, the screen-printed tri-electrode plate 114 further has a working electrode transfer portion 122 (electrically connected to the working electrode 116), a counter electrode transfer portion 124 (electrically connected to the counter electrode 118), and a reference electrode transfer portion 126 (electrically connected to the reference electrode 120), and may output the measured signal to a signal analysis device (not shown) to produce a potential-current diagram.

When the screen-printed tri-electrode plate 114 is used to detect the concentrations of the target heavy metal ions 102, the working electrode 116, the counter electrode 118, and the reference electrode 120 are immersed in the acid liquid 110 containing the target heavy metal ions 102 to detect the concentrations of the target heavy metal ions 102. When the material of the reference electrode 120 includes silver and the pH of the acid liquid 110 is less than 2, the acid liquid 110 severely corrodes the reference electrode 120, causing the detection function of the screen-printed tri-electrode plate 114 to fail.

Therefore, in order to prevent the acid liquid 110 from severely corroding the silver-containing reference electrode 120 and to effectively desorb the target heavy metal ions 102, the acid liquid 110 would have a pH between 2 and 5. As such, the target heavy metal ions 102 may weaken the bond between the ion-imprinted polymers 106 and the target heavy metal ions 102 and maintain the operation of the reference electrode 120.

In another embodiment, the anodic stripping voltammetry is performed to detect the concentrations of the target heavy metal ions 102 using, for example, a rod-shaped tri-electrode system 128. The device schematic of the rod-shaped tri-electrode system 128 is shown in part (b) of FIG. 3. The rod-shaped tri-electrode system 128 has a working electrode 130, a counter electrode 132, and a reference electrode 134. The material of the working electrode 130 includes gold or bismuth. The material of the counter electrode 132 includes gold or carbon. The material of the reference electrode 134 includes silver chloride or silver.

When the concentrations of the target heavy metal ions 102 are detected using the rod-shaped tri-electrode system 128, the working electrode 130, the counter electrode 132, and the reference electrode 134 are immersed in the acid liquid 110 containing the target heavy metal ions 102 to detect the concentrations of the target heavy metal ions 102 to produce a potential-current diagram.

It is to be noted that most of the surface of each of the counter electrode 132 and the reference electrode 134 of the rod-shaped tri-electrode system 128 is covered by a glass 136. Thus, when the material of the reference electrode 134 includes silver, the glass 136 may protect most of the reference electrode 134 from corrosion by the acid liquid 110. Therefore, as compared to the screen-printed tri-electrode plate 114, the rod-shaped tri-electrode system 128 is more resistant to acid. Therefore, the acid liquid 110 with a stronger acidity may be selected to achieve a more effective desorption of the target heavy metal ions 102 without damaging the reference electrode 134. The pH of the acid liquid 110 is, for example, between 0 and 5.

Conventional detection methods for heavy metal ions involve electrode modification to reduce the effect of the non-target object 103 on the detection results, but electrode modification is complex and expensive. As compared to the detection method involving a conventional electrode modification, the detection method of the disclosure is simple and cost-saving. Specifically, when the concentrations of the target heavy metal ions 102 are detected, the working electrodes 116/130, the counter electrodes 118/132, and the reference electrodes 120/134 may be directly used without electrode modification, thereby achieving a cost-saving effect.

Further, in the method of the disclosure, the interference matrix of the waste water is completely replaced with the acid liquid 110 before the concentrations of the target heavy metal ions are detected by the electrochemical method. In this way, the interference may be effectively reduced, and the oxidation potential of the target heavy metal ions is kept consistent with a pure water system, thereby effectively reducing error. In addition, in the disclosure, any matrix in raw water may be replaced with an acid liquid of a fixed concentration, which may effectively extend the service life of the electrodes.

Further, in the method of the disclosure, before the concentrations of the target heavy metal ions 102 are detected by the electrochemical method, since the non-target object 103 interfering with the detection is removed, interference from the non-target object during the detection may be eliminated, thereby obtaining more accurate detection results. More specifically, since the issue of the non-target object 103 interfering with the detection results is alleviated, by detecting the target heavy metal ions 102 with low concentrations via the method of the disclosure, the low-intensity signals thereof are not suppressed by the non-target object and are accordingly detected. The detection method of the disclosure may detect the target heavy metal ions 102 in concentrations of equal to or less than about 15 ppm, about 10 ppm, about 5 ppm, or about 2 ppm, for example.

Furthermore, after the acid liquid 110 of the disclosure desorbs the target heavy metal ions 102 in the ion-imprinted polymer tube 104, the target heavy metal ions 102 may be detected immediately without reprocessing the target heavy metal ions 102. In this way, the effect of reducing detection steps may be achieved.

Hereinafter, multiple experimental examples and comparative examples are provided to explain the effects of the above embodiments, but the scope of the disclosure is not limited to the following.

### EXPERIMENTAL EXAMPLE 1

A waste water with a pH of about 6 and a conductivity of about 2,000 microSiemens/cm was provided. Next, 2 ppm of copper ions was added to the waste water. Thereafter, the waste water was flowed through an ion-imprinted polymer tube having a copper ion-imprinted polymer to adsorb the copper ions.

The detailed preparation method of the copper ion-imprinted polymer is described in the following. 4 mmole of 4-vinyl pyridine (as a functional monomer) and 0.5 mmole of copper nitrate (Cu(NO₃)₂) (as template ions) were added in 35 ml of acetonitrile and stirred overnight. Then, 20 mmole of ethylene glycol dimethacrylate (EGDMA) (as a cross-linking agent) was added to the solution, N₂ was introduced in the solution to remove O₂ in the solution, the solution was placed in an oil bath at 65° C., and then 100 mg of azobisisobutyronitrile (AIBN) (as a starter) was added and stirred to react in the solution for 24 hours. After the reaction was completed, the resulting powder was first rinsed several times with methanol/water in a volume ratio of 1:4 to remove unreacted materials, and then rinsed several times with 0.5 M HCl to remove Cu^{2|} in the powder. The powder was then rinsed with deionized water until the neutral powder was obtained, and the powder was dried in an oven. Then, polishing was performed to obtain a copper ion-imprinted polymer.

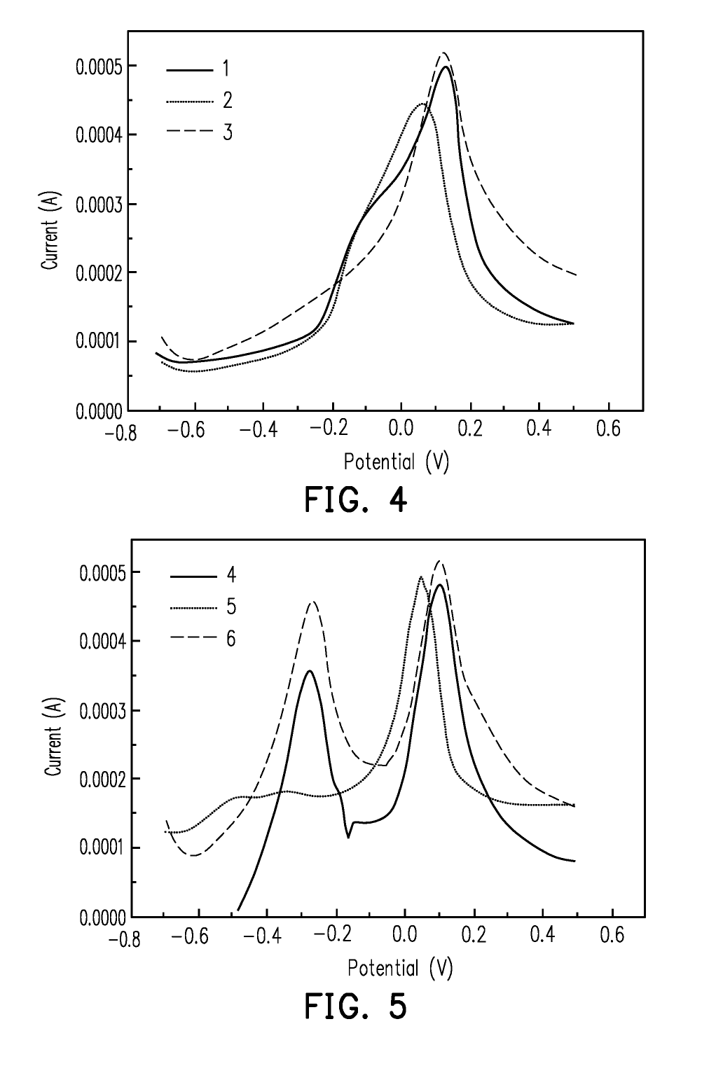

Next, the ion-imprinted polymer tube was rinsed with deionized water to remove the matrix. Then, the copper ions were desorbed using hydrochloric acid having a pH of about 2. Next, anodic stripping voltammetry was performed on the hydrochloric acid having the copper ions using a screen-printed tri-electrode plate. The results are shown in curve 1 of FIG. 4.

### COMPARATIVE EXAMPLE 1

The same waste water as experimental example 1 was provided. Next, 2 ppm of copper ions was added to the waste water. Thereafter, anodic stripping voltammetry was performed on the waste water directly using a screen-printed tri-electrode plate. The results are shown in curve 2 of FIG. 4.

### COMPARATIVE EXAMPLE 2

Deionized water was provided. 2 ppm of copper ions was added to the deionized water. Thereafter, anodic stripping voltammetry was performed on the deionized water directly using a screen-printed tri-electrode plate. The results are shown in curve 3 of FIG. 4.

Comparing curve 1, curve 2, and curve 3 of FIG. 4, it may be seen that the potential and current signals detected in experimental example 1 are very close to the detection results of comparative example 2. On the other hand, the potential and current signals detected in comparative example 1 are significantly shifted as compared with the detection results of comparative example 2. This shows that a more accurate quantitative and qualitative analysis may be achieved by the detection method of the disclosure.

Further, the concentrations of the copper ions measured in experimental example 1, comparative example 1, and comparative example 2 are recorded in Table 1. In addition, atomic absorption spectroscopy (AAS) was performed to detect the concentration of the copper ions via an atomic absorption spectrum apparatus. The results are also recorded in Table 1.

TABLE 1

| | Copper ion | Copper ion | |
|---|---|---|---|
| | concentration | concentration | |
| | (ppm) detected | (ppm) detected | |
| | by anodic | by atomic | |
| | stripping | absorption | Variation |
| | voltammetry | spectroscopy | (%) |
| Experimental example 1 | 1.81 | 1.98 | 6.7 |
| Comparative example 1 | 0.88 | 1.99 | 56.1 |
| Comparative example 2 | 1.98 | 1.98 | about 0 |

In Table 1, as compared to the copper ion concentration of comparative example 1 directly detected with anodic stripping voltammetry without treatments, the copper ion concentration of experimental example 1 measured by anodic stripping voltammetry after the adsorption, rinsing, and desorption treatments of the disclosure is closer to the actual concentration of the copper ions. In addition, comparative example 1 has a greater variation than experimental example 1, because the detection method for heavy metal ions of the disclosure may alleviate signal shift and suppression caused by the matrix, and may improve the accuracy of qualitative and quantitative detection results.

### EXPERIMENTAL EXAMPLE 2

A waste water with a pH of about 6 and a conductivity of about 2,000 microSiemens/cm was provided. Next, 2 ppm of lead ions was added to the waste water. Thereafter, the waste water was flowed through an ion-imprinted polymer tube having a lead ion-imprinted polymer to adsorb lead ions.

The detailed preparation method of the lead ion-imprinted polymer is described in the following. 0.89 g of 1-vinylimidazole and 0.0828 g of lead nitrate Pb(NO₃)₂ were added to 5 ml of tetrahydrofuran (THF) and stirred for 30 minutes. Then, 0.13 g of 3-(trimethoxysilyl) propylmethacrylate (TMSPMA) and 2 ml of tetrahydrofuran were added to the solution, N₂ was introduced in the solution to remove O₂ in the solution, the solution was placed in an oil bath at 68° C., and 1.6 mg of AIBN was added in the solution and stirred to react for 16 hours. After the reaction was completed, the resulting powder was first rinsed several times with 2M nitric acid to remove Pb²⁺in the powder. The powder was then rinsed with deionized water until the neutral powder was obtained, and the powder was dried in an oven. The product was obtained after polishing.

Next, the ion-imprinted polymer tube was rinsed with deionized water to remove the matrix. Then, lead ions were desorbed using hydrochloric acid having a pH of about 2. Next, anodic stripping voltammetry was performed on the hydrochloric acid having the lead ions using a screen-printed tri-electrode plate.

### COMPARATIVE EXAMPLE 3

The same waste water as experimental example 2 was provided. Next, 2 ppm of lead ions was added to the waste water. Thereafter, anodic stripping voltammetry was performed on the waste water directly using a screen-printed tri-electrode plate.

### COMPARATIVE EXAMPLE 4

Deionized water was provided. 2 ppm of lead ions was added to the deionized water. Thereafter, anodic stripping voltammetry was performed on the deionized water directly using a screen-printed tri-electrode plate.

Further, the concentrations of the lead ions measured in experimental example 2, comparative example 3, and comparative example 4 are recorded in Table 2. In addition, atomic absorption spectroscopy (AAS) was performed to detect the concentration of the lead ions via an atomic absorption spectrum. The results are also recorded in Table 2.

TABLE 2

| | Lead ion | Lead ion | |
|---|---|---|---|
| | concentration | concentration | |
| | (ppm) detected | (ppm) detected | |
| | by anodic | by atomic | |
| | stripping | absorption | Variation |
| | voltammetry | spectroscopy | (%) |
| Experimental example 2 | 1.85 | 1.97 | 6.1 |
| Comparative example 3 | 0.1 | 1.99 | 94.5 |
| Comparative example 4 | 1.97 | 1.98 | about 0 |

In Table 2, the lead ion concentration of experimental example 2 measured by anodic stripping voltammetry after the adsorption, rinsing, and desorption treatments of the disclosure is closer to the actual concentration of the lead ions. In contrast, the lead ion concentration in comparative example 3 is completely undetectable. It may be seen that the detection method for heavy metal ions of the disclosure may alleviate signal shift and suppression caused by the matrix, and may improve the accuracy of the qualitative and quantitative detection results.

### EXPERIMENTAL EXAMPLE 3

A copper ion-imprinted polymer (manufacturing method is the same as that of experimental example 1) and a lead ion-imprinted polymer (manufacturing method is the same as that of experimental example 2) were mixed evenly with silica sand (trade name: Aldrich 806765, particle size: 3 μm, purchased from Sigma Aldrich). Then, the mixture was filled in an ion-imprinted polymer tube. Next, a simulated waste water with a conductivity of about 2,400 microSiemens/cm was provided, and 10 ppm of copper ions and 10 ppm of lead ions were added to the simulated waste water.

Thereafter, the simulated waste water was flowed through the ion-imprinted polymer tube having the copper ion-imprinted polymer and the lead ion-imprinted polymer to adsorb copper ions and lead ions. Next, the ion-imprinted polymer tube was rinsed with deionized water to remove the matrix. Then, the copper ions and the lead ions were desorbed using hydrochloric acid having a pH of about 2. Next, anodic stripping voltammetry was performed using a screen-printed tri-electrode plate. The results are shown in curve 4 of FIG. 5.

### COMPARATIVE EXAMPLE 5

The same simulated waste water as experimental example 3 was provided. Next, 10 ppm of copper ions and 10 ppm of lead ions were added to the simulated waste water. Thereafter, anodic stripping voltammetry was performed on the waste water directly using a screen-printed tri-electrode plate. The results are shown in curve 5 of FIG. 5.

### COMPARATIVE EXAMPLE 6

10 ppm of copper ions and 10 ppm of lead ions were added to deionized water. Thereafter, anodic stripping voltammetry was performed on the deionized water directly using a screen-printed tri-electrode plate, and the results are shown in curve 6 of FIG. 5.

In FIG. 5, as compared to the results of comparative example 6, the potential and current signals of the simulated waste water of comparative example 5 without adsorbing, rinsing, and desorbing treatments are significantly shifted, in which the current signal of the lead ions (the left half of FIG. 5) is even completely suppressed. In contrast, the detection results of experimental example 3 are close to those of comparative example 6, indicating that in the detection method for heavy metal ions of the disclosure, the matrix in the waste water is replaced by hydrochloric acid, which may effectively solve the issue of reduced accuracy of quantitative and qualitative analysis of the target heavy metal ions caused by matrix interference to anodic stripping voltammetry.

In addition, FIG. 5 shows the obvious signals of copper ions and lead ions, thus indicating that two kinds of target heavy metal ions in the waste water to be tested are simultaneously adsorbed, rinsed, and desorbed by the detection method of the disclosure, and are then subjected to quantitative and qualitative analysis. Based on the above, the detection method for heavy metal ions of the disclosure may effectively alleviate the issue of signal shift and suppression caused by the matrix in the waste water, and may improve the accuracy of the qualitative and quantitative detection results. In addition, the detection method of the disclosure may effectively detect target heavy metal ions with low concentrations.

In addition, the detection method of the disclosure does not require electrode modification, and different kinds of target heavy metal ions in the waste water are simultaneously adsorbed, rinsed, and desorbed in order in a single ion-imprinted polymer tube, and then the concentrations of the target heavy metal ions are directly detected. Therefore, the effects of reducing detection steps and costs may be achieved.

It will be apparent to those skilled in the art that various modifications and variations may be made to the structure of the disclosed embodiments without departing from the scope or spirit of the disclosure. In view of the foregoing, it is intended that the disclosure cover modifications and variations of this disclosure provided they fall within the scope of the following claims and their equivalents.

What is claimed is:

1. A detection method for heavy metal ions, comprising:
flowing a waste water through an ion-imprinted polymer tube for adsorbing at least two kinds of target heavy metal ions;
rinsing the ion-imprinted polymer tube to remove a non-target object from the ion-imprinted polymer tube;
desorbing the target heavy metal ions in the ion-imprinted polymer tube by using an acid liquid; and
performing an electrochemical method to detect concentrations of the target heavy metal ions.

2. The detection method for the heavy metal ions of claim 1, wherein the at least two kinds of target heavy metal ions in the waste water are simultaneously adsorbed by the ion-imprinted polymer tube.

3. The detection method for the heavy metal ions of claim 1, wherein the ion-imprinted polymer tube has at least two kinds of ion-printed polymers.

4. The detection method for the heavy metal ions of claim 1, wherein a conductivity of the waste water is greater than or equal to 2,000 microSiemens/cm.

5. The detection method for the heavy metal ions of claim 1, wherein the ion-imprinted polymer tube is rinsed with a deionized water.

6. The detection method for the heavy metal ions of claim 1, wherein the target heavy metal ions comprise at least two kinds of lead ions, copper ions, chromium ions, nickel ions, zinc ions, and cadmium ions.

7. The detection method for the heavy metal ions of claim 1, wherein the acid liquid comprises a sulfuric acid, a hydrochloric acid, a nitric acid, or a combination thereof.

8. The detection method for the heavy metal ions of claim 7, wherein the acid liquid has a pH between 0 and 5.

9. The detection method for the heavy metal ions of claim 1, wherein the electrochemical method comprises detecting the concentrations of the target heavy metal ions using a rod-shaped tri-electrode system or a screen-printed tri-electrode plate.

10. The detection method for the heavy metal ions of claim 1, wherein after the acid liquid desorbs the target heavy metal ions in the ion-imprinted polymer tube, the concentrations of the target heavy metal ions are directly detected by the electrochemical method.

11. The detection method for the heavy metal ions of claim 1, wherein the electrochemical method comprises anodic stripping voltammetry or cyclic voltammetry.

12. The detection method for the heavy metal ions of claim 1, wherein the concentrations of the target heavy metal ions are equal to or less than 15 ppm.
